(19) United States
(12) Patent Application Publication
(10) Pub. No.: US 2015/0111916 A9
(43) Pub. Date: Apr. 23, 2015

(54) Treatment of pain using a composition of opioid/Toll-like receptor 4 antagonists and dextro enantiomers thereof

(71) Applicant: Annette Channa Toledano, North Miami, FL (US)

(72) Inventor: Annette Channa Toledano, North Miami, FL (US)

(21) Appl. No.: 13/851,267

(22) Filed: Mar. 27, 2013

Related U.S. Application Data
(63) Continuation-in-part of application No. 13/799,287, filed on Mar. 13, 2013.
Application No. 13/799,287 is a continuation-in-part of application No. 12/824,367, filed on Jun. 28, 2010.
(60) Provisional application No. 61/395,772, filed on May 17, 2010.
(60) Provisional application No. 61/343,489, filed on Apr. 29, 2010.

(51) Int. Cl. A61K 31/485

(52) U.S. Cl. CPC A61K 31/485
USPC 514/282; 514/279; 514/289; 514/456

(57) ABSTRACT

This invention relates to the use of a composition comprising opioid/Toll-like receptor 4 (TLR4) antagonists and dextro enantiomers thereof for the treatment of pain, where the opioid antagonists treat pain by blocking TLR4. More preferably, the dextro opioid/TLR4 antagonist enantiomer treats pain by blocking TLR4 while not affecting the opioid receptors and therefore avoiding adverse effects associated with blocking the opioid receptors. Examples of opioid antagonist and their dextro enantiomers include naltrexone, naloxone and nalmefene, and pharmaceutically acceptable salts thereof, pharmaceutical compositions thereof and their use in the treatment of pain, particularly neuropathic pain.

### CROSS-REFERENCE TO RELATED APPLICATIONS

This application is a continuation in part of U.S. application Ser. No. 13/799,287 filed Mar. 13, 2013, which is a continuation in part of U.S. application Ser. No. 12/824,367 filed Jun. 28, 2010, which claims the benefit of U.S. Provisional Application Ser. No. 61/343,489 filed on Apr. 29, 2010 and Provisional Application Ser. No. 61/395,772 filed on May 17, 2010, the contents of which are hereby both incorporated by reference in their entirety.

### FIELD OF INVENTION

This invention relates to the use of a composition of opioid/Toll-like receptor 4 antagonists and dextro enantiomers thereof in the treatment, prevention and reversal of pain.

### BACKGROUND

Various μ-opioid receptor ligands have been tested and were found to possess action as agonists or antagonists of Toll-like receptor 4 (TLR4). Toll-like receptors, found in the glia, are a class of receptors that play a key role in the innate immune system. They recognize pathogen-associated molecular patterns (PAMPs) such as lipopolysaccharide (LPS) that are expressed on infectious agents. The TLR4s mediate the production of cytokines necessary for the development of effective immunity.

Activation of TLR4 by opioid agonists such as morphine leads to downstream release of inflammatory modulators including TNF-α and interleukin-1. Constant low-level release of these modulators is thought to reduce the efficacy of opioid drug treatment with time and to be involved in both the development of tolerance to opioid analgesic drugs and in the emergence of side effects such as hyperalgesia and allodynia which can become problems following extended use of opioid drugs.

In view of the fact that opioid agonists such as morphine act as TLR4 agonists while opioid antagonists such as naloxone and naltrexone act as TLR4 antagonists, the instant invention relates to μ-opioid receptor ligand as ligand of TLR4 and contemplates that allodynia is caused by abnormal activation of TLR4. Blockage of TLR4 with an opioid antagonist will hence eliminate allodynia.

(−)-enantiomers of opioid antagonists are the natural enantiomers: they have affinity to the opioid receptors. The (+)-enantiomers, also referred to as dextro-enantiomers, are the unnatural enantiomers, unlike natural enantiomers, dextro enantiomers have no significant affinity for opioid receptors. Both the (−)-enantiomers and the dextro enantiomers have been discovered to have affinity to TLR4, selecting the dextro enantiomers of opioid antagonists will selectively antagonize only TLR4 without affecting the opioid receptors. Blocking TLR4 will negate the release of inflammatory mediators.

It is well established in medical literature that treatments currently available for pain have limitations. Opioid drugs cause tolerance, dependence and side effects sufficiently serious to prompt recent action by the FDA to further restrict the drugs. NSAIDs, taken for a prolonged periods of time, are known to cause gastro-intestinal bleeding as well as toxicity to the liver, kidneys and other organs. Newly approved treatments, like the calcium channel alpha-2-delta ligands gabapentin and pregabalin and the serotonin and norepinephrine reuptake inhibitors milnacipran and duloxetine, require high doses to show nominal effectiveness, have a high dropout rate and carry many side effects.

In essence the instant invention recognizes that opioid receptor antagonists exert their action in a site other than the opioid receptors. That site is the immune system receptor TLR4 located on glia cells. The invention recognizes that the immune system going awry is the cause of neuropathic pain. Blocking TLR4s with an opioid receptor antagonist, more preferably with a dextro enantiomer of an opioid receptor antagonist which does not affect the opioid receptors, is the mainstay of this invention. Blocking TLR4s with a dextro enantiomer of an opioid receptor antagonist solves the perplexing problem of neuropathic pain. The instant invention is based on findings from two double-blind placebo-controlled clinical trial of 78 subjects treated with the opioid receptor naltrexone which proved the efficacy of this treatment for pain.

This invention is a novel approach to the treatment of chronic pain aimed at breaking the cycle of allodynia, or “memory pain”, as allodynia may be described in lay terms. Allodynia is a clinical feature of many painful conditions such as neuropathies. The specific composition of drugs and the dosage needed to create that effect is the subject of the instant invention. A hallmark of this invention consists of the realization that the origin of neuropathic pain is due to abnormal activation of TLR4 which trigger a pro-inflammatory cascade. That cascade is interrupted and in most cases eliminated by administration of a composition of opioid/TLR4 antagonist, more preferably, the instant invention teaches that opioid/TLR4 antagonist dextro enantiomers inhibits TLR4 without affecting the opioid receptors avoiding related adverse effect.

Adverse effects caused by blockage of the opioid receptors differ in a low dose compared to the regular dose: in a low dose they include but are not limited to anxiety, aggressiveness, irritability and insomnia, while the regular dose hinders the ability to experience pleasure. Use of opioid/TLR4 antagonist dextro enantiomer which does not interact with the opioid receptors will eliminate those adverse effects.

Moreover, the methods provided by the instant invention consist of administering to a mammal an effective amount of a composition of opioid/TLR4 or dextro enantiomeric mixtures thereof or a pharmaceutically acceptable salt thereof for the treatment, prevention and reversal of pain.

The instant invention is based in part on findings from a clinical trial performed on behalf of the invention. The findings are sighted in U.S. patent application Ser. No. 13/799,287. The study found that the opioid antagonist naltrexone in combination with the direct acting alpha-2 adrenergic agonist clonidine was effective in treating pain. The direct acting alpha-adrenergic agonist clonidine was added to the opioid/TLR4 antagonist naltrexone to abate naltrexone's adverse effects cause by its antagonism of the opioid receptors. Although, clonidine in the combination added to the pain treatment effect of naltrexone, the instant invention contemplates that naltrexone alone can induce sufficient pain treatment effect especially when used in the form of dextro naltrexone. Dextro naltrexone has a direct action against the pain producing receptors TLR4 without affecting the opioid receptors.

The instant invention discloses use of the opioid antagonist naltrexone and dextro naltrexone and appropriate mixtures thereof for the treatment of pain, wherein, the dextro enantiomer of the opioid antagonist treat pain by blocking Toll-like receptor 4 (TLR4) while not affecting the opioid receptors which caused the adverse effects. The use of the opioid antagonist dextro enantiomer which antagonizes only the TLR4 without antagonizing the opioid receptors eliminates the concern with opioid antagonist's adverse effects and allow for their use in the treatment of pain.

Related aspect of this invention are described in the Applicant's U.S. patent application Ser. Nos. 12/824,367, 13/799,287, 13/837,099 and 13/841,110 which are incorporated herein by reference.

It has been suggested in the literature that low dose naltrexone (less than 25 mg/day) exerts the opposite effect of naltrexone in full dose. In other words it has a bimodal dose response. While naltrexone in the full dose blocks the opiate system and reduces the effect of endogenous endorphins and exogenous morphine, low dose naltrexone promotes the production of endorphins by the known mechanism of up regulation caused by partial blockage of the opiate receptor. The beneficial effect of low dose naltrexone was attributed to the increase in natural pain relief effect of endorphins. The beneficial effect of low dose naltrexone can be better explained by its action on TLR4.

The pain treatment effect of the opioid antagonists on TLR4 is correlated to the dose. In the instant invention clinical trial, the pain treatment effect of the opioid antagonist, Naltrexone was observed starting in a low dose, which is effective in most cases, and increased with a higher dose. The pain treatment effect was accomplished in doses lower than those prescribed for addiction control.

The instant invention contemplates usage of an opioid receptor antagonist for example naltrexone for the treatment, prevention and reversal of pain in an amount consistent with a “low” dose (25 mg or less/day).

A low dose naltrexone study by Ploesser et al. showed double digit incidence of nervousness, insomnia, and anxiety. The increase in endorphin levels may be the reason why it was found that low dose naltrexone has a double digit rate of adverse reactions such as alertness, nervousness, anxiety and insomnia as contrasted with the full dose, the full dose of naltrexone has a single digit incidence of alertness, nervousness, anxiety, and insomnia. The full dose of naltrexone carries adverse effects of diminished libido and diminished ability to experience pleasure from food and other activities.

The instant invention teaches that administration of opioid antagonists for the treatment of pain is effective in a low dose as well as in a high dose. This was demonstrated in the instant invention's clinical trials. Using the opioid receptor antagonist naltrexone, it was demonstrated that it dose-dependently treats pain. Regarding the issue of the opioid receptor antagonist adverse effects, based on the mechanism of action, an innovative way to avoid adverse effects that arise from their action on the morphine receptors is to exclusively use their dextro enantiomers or a mixture thereof which act only on TLR4 and do not act on the morphine receptors.

The instant invention, example 1 demonstrates a case of a human subject who used naltrexone in a low dose of 2 mg twice daily for cervical pain. The subject experienced a significant relief of his pain however, he discontinued using the drug after 2 weeks because he was unable to tolerate the side effects that included anxiety and irritability he stated that he did not feel “normal” and “did not want to do anything”. The subject of example used 12.5 mg of naltrexone daily for cervical pain, he too experienced relief of his pain but elected to discontinue the treatment after 10 days because of adverse effects, at this medium dose, naltrexone caused interference with his ability to experience pleasure, particularly from food and alcohol in addition his libido was reduced, he states that he felt “blah”.

This teaches that in a low dose the side effects of naltrexone are associated with the “up regulation” of the opioid receptors and additional release of endorphins. In the full dose, the side effects of naltrexone are associated with the blockage of the opioid receptors, causing diminished ability to experience pleasure. This is exemplified in the Examples referred to above.

This invention teaches that the use of dextro enantiomers of opioid antagonist is advantageous; the dextro opioid antagonists treat pain by blocking (TLR4) while not affecting the opioid receptors.

Other known opioid receptor antagonist and their dextro enantiomers used in clinical or scientific practice can also be used for the treatment of pain. They include but are not limited to the following: naloxone, nalmefene, norbinaltorphimine, nalorphine, methylnaltrexone, samidorphan, cyprodime, naltrindole, amentoflavone, naltriben, norbinaltorphimine, and the naltrexone metabolite 6-beta-naltrexol.

Allodynia is a clinical feature of many painful conditions, such as back pain, chronic pain, neuropathic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis. It becomes apparent that allodynia plays a role in every kind of pain.

The instant invention offers a new explanation for the occurrence of allodynia, or “memory pain”, connecting the dots of existing knowledge from animal model studies along with the vast information gleaned from the instant invention clinical trials, it is now evident that allodynia is caused by abnormal endogenous activation of TLR4 that in turn trigger a pro-inflammatory cascade. The instant invention's clinical trial for back pain verified that the pain is interrupted by the opioid/TLR4 antagonist naltrexone. The instant invention claims that the opioid receptors dextro enantiomers offer a more specific blockage and therefore avoid adverse effects.

The instant invention's clinical trial provides proof to a number of novel claims made by this invention, starting with the first time proof in a double-blind placebo-controlled clinical trial that the opioid/TLR4 antagonist naltrexone treats pain. Additionally, the clinical trial showed naltrexone was effective in the treatment of acute migraine headache, trigeminal neuralgia, vulvodynia and irritable bowel syndrome. Although the study used a combination of opioid/TLR4 antagonist with clonidine it is felt that the contribution of clonidine to the pain treatment effect was about 20-30% of the total effect. Another claim the instant invention makes involves reducing adverse effect from the opioid antagonists when treating pain, side effects can be reduced, either by adding clonidine to abate these effects, or by using only the dextro enantiomers of the opioid antagonists.

In addition to treating neuropathic pain, TLR4 antagonism can play a role in treating nociceptive pain, presumably by affecting the allodynic component of nociceptive pain. In the clinical trial, subjects with nociceptive pain, such as joint pain either from tendinitis or from arthritis, had improvement of their nociceptive pain after treatment with the opioid antagonist naltrexone.

Contrary to opioid pain medications, the opioid antagonists are primarily non-addictive medications.

Based upon this, the instant invention first teaches the use of an opioid/TLR4 antagonist, particularly naltrexone for its antagonism of the TLR4 and blocking release of inflammatory modulators. Secondly, the invention teaches use of dextro enantiomers of the opioid/TLR4 antagonist naltrexone to avoid the adverse effects of naltrexone. In addition to naltrexone, the invention contemplates several other forms of dextro opioid antagonists selected from a group consisting of naloxone, nalmefene, norbinaltorphimine, nalorphine, methylnaltrexone, samidorphan, cyprodime, naltrindole, amentoflavone, naltriben, norbinaltorphimine, and metabolite 6-beta-naltrexol and 6-alpha-naltrexol and other metabolites and pro drugs thereof, including all enantiomeric and epimeric forms as well as the appropriate mixtures thereof, or pharmaceutically acceptable salts or solvates of any thereof.

### DESCRIPTION OF EMBODIMENTS

The present invention provides methods for the treatment, prevention and reversal of pain in a mammal suffering from pain by administering to said mammal a therapeutically effective amount of a composition comprising an opioid/TLR4 antagonist that blocks TLR4.

Furthermore, the present invention provides methods for treatment, prevention and reversal of pain in a mammal suffering from pain by administering to that mammal a therapeutically effective amount of a composition predominantly comprising the dextro enantiomer of an opioid/TLR4 antagonist that blocks TLR4 without affecting the opioid receptors.

In a preferred embodiment, the present invention provides a method for treatment, prevention and reversal of pain in a mammal suffering from pain by administering to the mammal a composition comprising a therapeutically effective amount of an opioid/TLR4 antagonist or a dextro enantiomer or racemic mixture thereof, or a pharmaceutically acceptable salt or solvate thereof.

In a preferred embodiment, the invention offers a method for treatment, prevention and reversal of pain in a mammal by administering a composition comprising a therapeutically effective amount of an opioid/TLR4 antagonist. The opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-beta-naltrexol and 6-alpha-natrexol metabolites thereof, including all enantiomeric and epimeric forms as well as the appropriate mixtures thereof, as well as pro drugs or metabolites thereof or pharmaceutically acceptable salts or solvates of any thereof.

In a preferred embodiment, the present invention provides a method for the treatment, prevention and reversal of pain in a mammal by administering to said mammal a composition comprising a therapeutically effective amount of naltrexone, naloxone or nalmefene, or dextro enantiomer or racemic mixture thereof or a pharmaceutically acceptable salt of any thereof.

In a preferred embodiment, the present invention provides a method for the treatment, prevention and reversal of pain in a mammal by administering to said mammal a composition comprising of a therapeutically effective amount an effective amount of dextro naltrexone mixture or a pharmaceutically acceptable salt thereof.

In a preferred embodiment, the present invention provides a method for the treatment, prevention and reversal of pain in a mammal by administering to said mammal a composition comprising a therapeutically effective amount of dextro naloxone mixture or a pharmaceutically acceptable salt thereof.

In a preferred embodiment, the present invention provides a method for the treatment, prevention and reversal of pain in a mammal by administering to said mammal a composition comprising a therapeutically effective amount of dextro nalmefene mixture or a pharmaceutically acceptable salt thereof.

In a particular embodiment the amount of the opioid/TLR4 antagonist varies from about 0.004 mg/kg to about 4.3 mg/kg, preferably from about 0.004 mg/kg to about 0.71 mg/kg, and most preferably from about 0.004 mg/kg to about 0.21 mg/kg.

In a particular embodiment the amount of naltrexone varies from about 0.004 mg/kg to about 4.3 mg/kg, preferably from about 0.004 mg/kg to about 0.71 mg/kg, and most preferably from about 0.004 mg/kg to about 0.21 mg/kg.

In a particular embodiment the amount of dextro naltrexone varies from about 0.004 mg/kg to about 4.3 mg/kg, preferably from about 0.004 mg/kg to about 0.71 mg/kg, and most preferably from about 0.004 mg/kg to about 0.21 mg/kg.

In a particular embodiment the present invention provides a method wherein the opioid antagonist/TLR4 is naltrexone in a sustained release formulation, as well as pro drugs thereof or any enantiomeric and epimeric forms thereof, as well as the appropriate mixtures thereof, or pharmaceutically acceptable salts or solvates of any thereof.

In a particular embodiment the present invention provides a method wherein the opioid antagonist/TLR4 is dextro naltrexone in a sustained release formulation, as well as pro drugs thereof or any enantiomeric and epimeric forms thereof, as well as the appropriate mixtures thereof, or pharmaceutically acceptable salts or solvates thereof.

In a particular embodiment the present invention provides a method wherein the opioid antagonist comprises a therapeutically active enantiomer, the dextro enantiomer that has less than about 50%, less than about 40%, less than about 30%, less than about 20%, less than about 10%, less than about 5% or less than about 1% by weight of other enantiomers.

In a particular embodiment the present invention provides a method wherein the opioid antagonist comprises a therapeutically active enantiomer compound primarily in the dextro form, which can be from greater than 50% to 100% dextro enantiomer. Compositions comprising primarily the dextro form of an opioid antagonist can have greater than 50% to 60% dextro enantiomer, but preferably have greater than 60% to 70% dextro enantiomer, more preferably greater than 70% to 80% dextro enantiomer, still more preferably greater than 80% to 90% dextro enantiomer, and most preferably, more than 90% dextro enantiomer.

In a particular embodiment the present invention provides a method for treatment of pain wherein the human dose range of naltrexone, or a pharmaceutically acceptable salt or solvate thereof, varies from about 0.25 mg-50 mg per day, preferably from about 0.25 mg-25 mg per day, most preferably from about 0.25 mg-15 mg per day, wherein said dose is formulated into a single dosage form.

In a particular embodiment the present invention provides a method wherein the single dosage form is administered once, twice, three or four times through the day.

In a particular embodiment the present invention provides a method wherein a therapeutically effective dose is administered systemically, via routes of mucosal, nasal, oral, parenteral, gastrointestinal, topical or sublingual routes.

In a particular embodiment the present invention provides a method wherein said composition is in a single dosage form, and said single dosage form is in the form of tablets, lozenges, troches, hard candies, liquid, powders, sprays, creams, salves and suppositories.

In a particular embodiment the present invention provides a method wherein the pharmaceutical composition is used for the treatment, prevention and reversal of neuropathic pain, back pain, chronic pain, diabetic neuropathic pain, trigeminal neuralgia pain, phantom limb pain, complex regional pain syndrome pain, acute herpetic pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis.

In a particular embodiment the present invention provides a method wherein the pharmaceutical composition is used for the treatment, prevention and reversal of nociceptive pain with an allodynic component.

In a particular embodiment the present invention provides a method wherein the composition containing a therapeutically effective amount of an opioid/TLR4 antagonist, or a pharmaceutically acceptable salt or solvate thereof, may optionally be administered with one or more other pharmacologically active agents. Appropriate optional agents include: non-steroidal anti-inflammatory drugs (NSAIDs), e.g. aspirin, ibuprofen, naproxen, naprosyn, diclofenac, ketoprofen, tolmetin, sulindac, mefanamic acid, meclofenamic acid, diflunisal, flufenisal, piroxicam, sudoxicam, isoxicam, celecoxib, fofecoxib, flosulide, meloxicam, 6-methoxy-2-naphthylacetic acid, nabumetone, nimesulide, steroidal anti-inflammatory drugs, tricyclic antidepressants (TCAs), selective serotonin reuptake inhibitors (SSRIs), serotonin-norepinephrine reuptake inhibitors (SNRIs), anticonvulsants, muscle relaxants, drugs with NMDA antagonist properties, tetrahydrocannabinol derivatives, antitussive, expectorants, decongestants, or antihistamines.

In a particular embodiment the present invention provides a method wherein, for non-human animal administration the term “pharmaceutical” as used herein may be replaced by “veterinary”.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete and will fully convey the scope of the invention to those skilled in the art.

### Pharmaceutical Composition

Enantiomers are two stereoisomers that are related to each other by a reflection: they are mirror images of each other, which are non-superimposable. Two compounds that are enantiomers of each other have the same physical properties, except for the direction in which they rotate polarized light and how they interact with different optical isomers of other compounds. As a result, different enantiomers of a compound may have substantially different biological effects. In nature, only one enantiomer of most chiral biological compounds is present.

Stereo selective reduction of naltrexone to alpha-naltrexol using tri-sec-butylborohydride has been described by Malspeis et al. Malspeis et al. (1975) Res. Commum. Chem. Pathol. Pharmacol. 12(1):43-65. As described there, stereo selective reduction of naltrexone and naloxone gave a mixture (85:15) of the 6 alpha and 6 beta hydroxy epimers, alpha and beta naltrexol and alpha and beta naloxol, respectively.

Substituting naltrexone with dextro naltrexone would have many advantages including more specific action on TLR4 and lack of antagonism of morphine receptors.

Preferred compounds to be used in the treatment of pain include the following opioid antagonist and their dextro enantiomers, naltrexone, naloxone, nalmefene, norbinaltorphimine, nalorphine, methylnaltrexone, samidorphan, cyprodime, naltrindole, amentoflavone, naltriben, norbinaltorphimine, and the naltrexone metabolite 6-beta-naltrexol.

Further compounds which can be used for the treatment of pain are the dextro enantiomers of the following opioid antagonists: naltrexone, naloxone, nalmefene, norbinaltorphimine, nalorphine, methylnaltrexone, samidorphan, cyprodime, naltrindole, amentoflavone, naltriben, norbinaltorphimine, and the naltrexone metabolite 6-beta-naltrexol.

The clinical trials demonstrated that adverse effects were minimal and transient with no indication of addiction. There were no reports of tolerance or withdrawal symptoms.

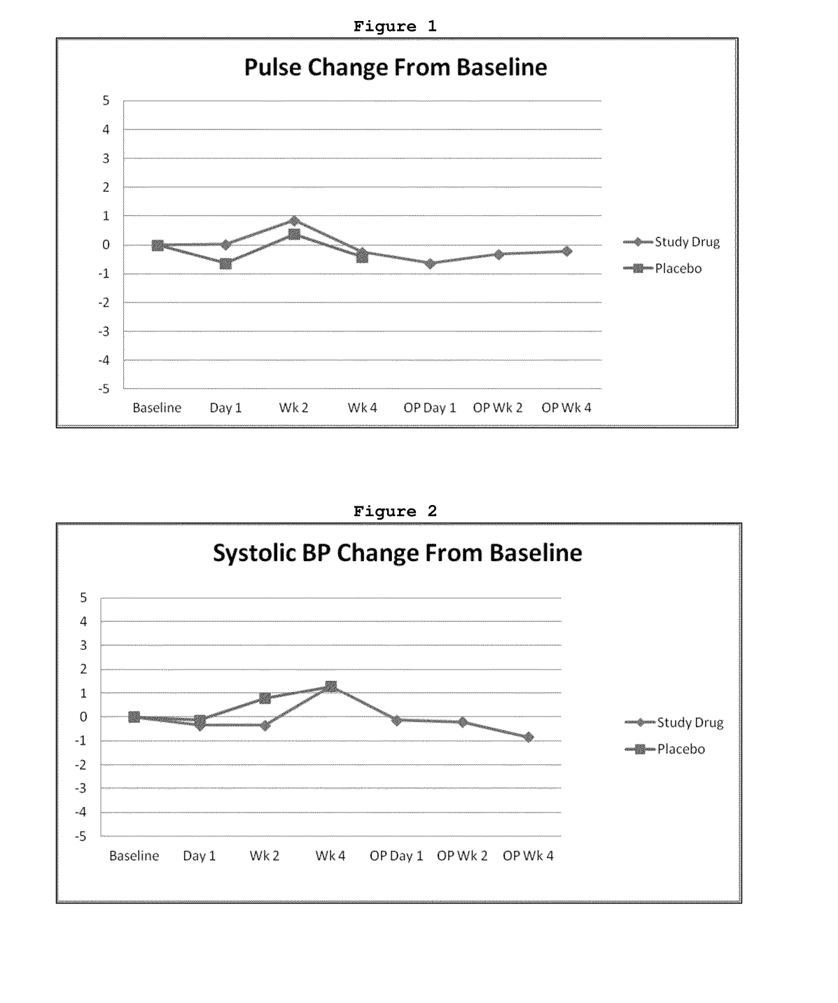

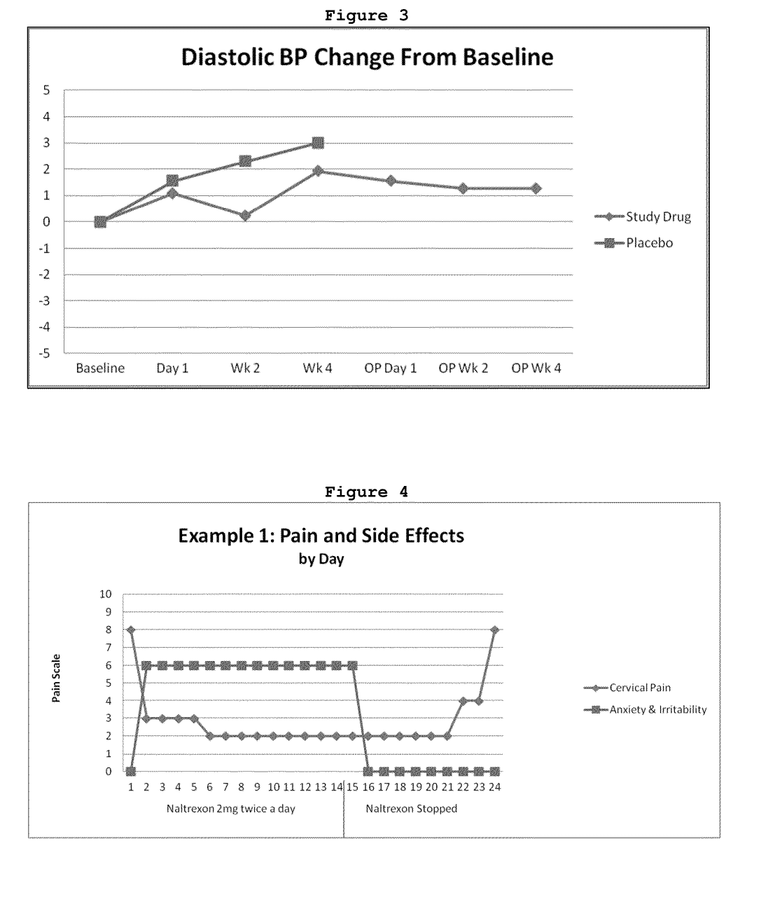

The following tables and graphs demonstrate safety of naltrexone for the treatment of pain. The data was obtained from a subject clinical trial conducted on behalf of this invention where naltrexone 2.25 mg/clonidine 0.025 mg were administered twice daily. Table 1 tabulates the change of the pulse from baseline by visit of the study drug group and the placebo group. The drawing in FIG. 1 graphs the change in the pulse from baseline of the study drug group versus the placebo group. Table 2 tabulates the change of the systolic blood pressure (BP) from baseline by visit of the study drug group and the placebo group. The drawing in FIG. 2 graphs the change in the systolic blood pressure (BP) from baseline of the study drug group versus the placebo group. Table 3 tabulates the change of the diastolic blood pressure (BP) from baseline by visit of the study drug group and the placebo group. The drawing in FIG. 3 graphs the change in the diastolic blood pressure (BP) from baseline of the study drug group versus the placebo group The data shows that there were no clinically significant changes in pulse, systolic blood pressure and diastolic blood pressure over a three to four week treatment, either during treatment period or Open Phase (OP).

TABLE 1
Pulse Change from Baseline by Visit
Pulse Change from Baseline

| | Base- | | | | OP | OP | OP |
|---|---|---|---|---|---|---|---|
| | line | Day 1 | Wk 2 | Wk 4 | Day 1 | Wk 2 | Wk 4 |
| Study | 0 | 0.0154 | 0.846 | −0.25 | −0.636 | −0.316 | −0.211 |
| Drug | | | | | | | |
| Mean | | | | | | | |
| Study | 26 | 26 | 26 | 24 | 22 | 19 | 19 |
| N | | | | | | | |
| Pla- | 0 | −0.636 | 0.37 | −0.417 | | | |
| cebo | | | | | | | |
| Mean | | | | | | | |
| Pla- | 27 | 27 | 27 | 24 | | | |
| cebo | | | | | | | |
| N | | | | | | | |

TABLE 2
Systolic BP Change from Baseline by Visit
Systolic Change From Baseline

| | Base- | | | | OP | OP | OP |
|---|---|---|---|---|---|---|---|
| | line | Day 1 | Wk 2 | Wk 4 | Day 1 | Wk 2 | Wk 4 |
| Study | 0 | −0.346 | −0.346 | 1.28 | −0.136 | −0.211 | −0.842 |
| Drug | | | | | | | |
| Mean | | | | | | | |
| Study N | 26 | 26 | 26 | 24 | 22 | 19 | 19 |
| Placebo | 0 | −0.136 | 0.778 | 1.29 | | | |
| Mean | | | | | | | |
| Placebo | 27 | 27 | 27 | 24 | | | |
| N | | | | | | | |

TABLE 3
Diastolic Change from Baseline by Visit
Diastolic BP Change From BL

| | Base- | | | | OP | OP | OP |
|---|---|---|---|---|---|---|---|
| | line | Day 1 | Wk 2 | Wk 4 | Day 1 | Wk 2 | Wk 4 |
| Study | 0 | 1.08 | 0.231 | 1.92 | 1.55 | 1.263 | 1.263 |
| Drug | | | | | | | |
| Mean | | | | | | | |
| Study N | 26 | 26 | 26 | 24 | 22 | 19 | 19 |
| Placebo | 0 | 1.55 | 2.296 | 3 | | | |
| Mean | | | | | | | |
| Placebo | 27 | 27 | 27 | 24 | | | |
| N | | | | | | | |

### EXAMPLES

The following examples demonstrate adverse effects in two human subjects who were dosed with naltrexone alone: their experience validates the need to use the dextro enantiomer of naltrexone because of the side effects they experienced.

### Example 1

Example 1 demonstrates a case of a human subject who used naltrexone in a low dose of 2 mg twice daily for cervical pain. The subject experienced a significant relief of his pain however he discontinued using the drug after 2 weeks because he was unable to tolerate the side effects that included anxiety and irritability.

Table 4 tabulates the change of pain and side effects for example 1 (on Naltrexone 2 mg twice daily) by day. The drawing in FIG. 4 graphs the change of pain and side effects for example (on Naltrexone 2 mg twice daily) by day. Table 4 and FIG. 4 show naltrexone's effect on pain and side effects. The pain treatment effect of naltrexone continued for a few days after discontinuation. The side effects resolved within one day of treatment discontinuation.

TABLE 4
Pain and Side Effects
By Day (Naltrexone 2 mg/day twice daily)

| Day | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| Cervical Pain | 8 | 3 | 3 | 3 | 3 | 2 | 2 | 2 | 2 | 2 | 2 | 2 | 2 | 2 | 2 | 2 | 2 | 2 | 2 | 2 | 2 | 4 | 4 | 8 |
| Anxiety & | 0 | 6 | 6 | 6 | 6 | 6 | 6 | 6 | 6 | 6 | 6 | 6 | 6 | 6 | 6 | 0 | 0 | 0 | 0 | 0 | 0 | 0 | 0 | 0 |
| Irritability | | | | | | | | | | | | | | | | | | | | | | | | |
| Naltrexone | 0 | 4 | 4 | 4 | 4 | 4 | 4 | 4 | 4 | 4 | 4 | 4 | 4 | 4 | 4 | 0 | 0 | 0 | 0 | 0 | 0 | 0 | 0 | 0 |
| Daily Dose | | | | | | | | | | | | | | | | | | | | | | | | |

### Example 2

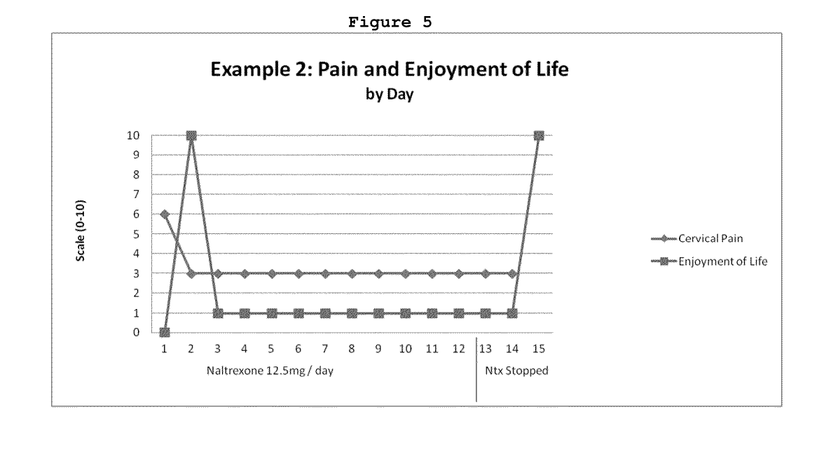

The subject of Example 2 used 12.5 mg per day of naltrexone for cervical pain. Table 5 tabulates the change of pain and enjoyment of life for example 2 (on Naltrexone 12.5 mg/day) by day. The drawing in FIG. 5 graphs the change of pain and enjoyment of life for example 2 (on Naltrexone 12.5 mg/day) by day. As with the subject in Example 1, he too experienced relief of his pain, but Example 2 elected to discontinue the treatment after 10 days because of adverse effects. At this medium dose, naltrexone caused interference with his ability to experience pleasure, particularly from food and alcohol. In addition, his libido was reduced, and he stated that he felt “blah”.

TABLE 5
Example 2 Pain and Side Effects
By Day (Naltrexone 12.5 mg/day)

| Day | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| Cervical Pain 6 | 3 | 3 | 3 | 3 | 3 | 3 | 3 | 3 | 3 | 3 | 3 | 3 | 3 | |
| Enjoyment | 10 | 1 | 1 | 1 | 1 | 1 | 1 | 1 | 1 | 1 | 1 | 1 | 1 | 10 |
| of Life | | | | | | | | | | | | | | |
| Naltrexone | 12.5 | 12.5 | 12.5 | 12.5 | 12.5 | 12.5 | 12.5 | 12.5 | 12.5 | 12.5 | 12.5 | 12.5 | 0 | 0 |
| Daily Dose | | | | | | | | | | | | | | |

### CITATION LIST

Bowling, Allen C. “Low-dose naltrexone (LDN) The “411” on LDN”. National Multiple Sclerosis Society. Retrieved 6 Jul. 2011.
Breivik H, Borchgrevink P C, Allen S M, Rosseland L A, Romundstad L, Hals E K, Kvarstein G, Stubhaug A. Assessment of pain. Br J. Anaesth. 2008; 101(1):17-24.doi:10.1093/bja/aen103. PMID 18487245.
Brine et al., “Ring C Conformation of 6.beta.-Naltrexol and 6.alpha.-Naltrexol Evidence from Proton and Carbon-13 NMR,” J. Org. Chem., (1976) 41(21):3445-3448.
Chatterjie et al., “Stereospecific synthesis of the 6.beta.-hydroxy metabolites of naltrexone and naloxone,” J. Med. Chem., Am. Chemical Society, (1975) 18(5):490-492.
Eur J Neurosci TNFα Levels and Macrophages Expression Reflect an Inflammatory Potential of Trigeminal Ganglia in a Mouse Model of Familial Hemiplegic Migraine Nat Rev Neurosci. 2009 January; 10(1): 23-36.
Hahn, et al., “Narcotic Antagonists. V. Stereochemistry of Reactions at C-6 in 14-Hydroxynoroxymorphone Derivatives,” J. Org. Chem., (1975) 40(1):31-34.
Harris et al., Effect of Pegylation on Pharmaceuticals, Nature, 2, p. 214-221 (2003).
Hutchinson M R, Coats B D, Lewis S S, Zhang Y, Sprunger D B, Rezvani N, Baker E M, Jekich B M, Wieseler J L, Somogyi A A, Martin D, Poole S, Judd C M, Maier S F, Watkins L R (November 2008). “Proinflammatory cytokines oppose opioid induced acute and chronic analgesia”. Brain, Behavior, and Immunity 22 (8): 1178-89. doi:10.1016/j.bbi.2008.05.004. PMC 2783238.PMID 18599265.
Hutchinson M R, Lewis S S, Coats B D, Rezvani N, Zhang Y, Wieseler J L, Somogyi A A, Yin H, Maier S F, Rice K C, Watkins L R (May 2010). “Possible involvement of Toll-Like Receptor 4/MD-2 activity of opioid inactive isomers causes spinal proinflammation and related behavioral consequences”. Neuroscience 167 (3): 880-93. doi:10.1016/j.neuroscience.2010.02.011.PMC 2854318. PMID 20178837.
Hutchinson M R, et al. Opioid-induced glial activation: mechanisms of activation and implications for opioid analgesia, dependence and reward. Sci. World J. 2007; 7:98-111.
Hutchinson M R, et al. Non-stereoselective reversal of neuropathic pain by naloxone and naltrexone: involvement of toll-like receptor 4 (TLR4) Eur. J. Neurosci. 2008; 28:20-.
Hutchinson M R, Lewis S S, Coats B D, Rezvani N, Zhang Y, Wieseler J L, Somogyi A A, Yin H, Maier S F, Rice K C, Watkins L R (May 2010). “Possible involvement of Toll-Like Receptor 4/MD-2 activity of opioid inactive isomers causes spinal proinflammation and related behavioral consequences”. Neuroscience 167 (3): 880-93.doi:10.1016/j.neuroscience.2010.02.011. PMC 2854318. PMID 20178837.
Hutchinson M R, et al. Proinflammatory cytokines oppose opioid-induced acute and chronic analgesia. Brain Behav. Immun. 2008 Jul. 1.
Hutchinson M R, et al. Minocycline supresses morphine-induced respiratory depression, supresses morphine-induced reward, and enhances systemic morphine-induced analgesia. Brain Behay. Immun. 2008 Jul. 31.
International Preliminary Exam Report on Patentability corresponding to PCT/US2007/009761, dated Oct. 28, 2008. cited by o
International Search Report corresponding to PCT/US2007/009761, dated Aug. 5, 2008.
Johnston I N, et al. A role for pro-inflammatory cytokines and fractalkine in analgesia, tolerance and subsequent pain facilitation induced by chronic intrathecal morphine. 2004; 24:7353-7365.
Komatsu T, Sakurada S, Katsuyama S, Sanai K, Sakurada T (2009). “Mechanism of allodynia evoked by intrathecal morphine-3-glucuronide in mice”. International Review of Neurobiology 85: 207-19. doi:10.1016/50074-7742(09)85016-2.PMID 19607972.
Lewis S S, Hutchinson M R, Rezvani N, Loram L C, Zhang Y, Maier S F, Rice K C, Watkins L R (January 2010). “Evidence that intrathecal morphine-3-glucuronide may cause pain enhancement via toll-like receptor 4/MD-2 and interleukin-1β”. Neuroscience 165 (2): 569-83. doi:10.1016/j.neuroscience.2009.10.011.PMC 2795035. PMID 19833175.
Malspeis et al., “Metabolic Reduction of Naltrexone I. Synthesis, Separation and Characterization of Naloxone and Naltrexone . . . ,” Res. Commun. Chem. Pathol. Pharmacol., 12(1), p. 43-65, (1975).
Mannelli P, Gottheil E, Van Bockstaele E J (2006). “Antagonist treatment of opioid withdrawal translational low dose approach”. J Addict Dis 25 (2): 1-8.doi:10.1300/J069v25n02_{—}01. PMID 16785213.
Milligan, E. D., and Watkins, L. R., Pathological and protective roles of glia in chronic pain, Nature Neuroscience Reviews, 10 (2009) 23-36.
Nasu-Tada K, Koizumi S, Tsuda M, Kunifusa E, Inoue K. Possible involvement of increase in spinal fibronectin following peripheral nerve injury in upregulation of microglial P2×4, a key molecule for mechanical allodynia. Glia. 2006; 53:769-775.
Ngian G S, Guymer E K, Littlejohn G O (February 2011). “The use of opioids in fibromyalgia” (PDF). Int J Rheum Dis 14 (1): 6-11. doi:10.1111/j.1756−185×.2010.01567.x. PMID 21303476.
Novella, Steven. “Low Dose Naltrexone—Bogus or Cutting Edge Science?”. Retrieved 5 Jul. 2011.
Obata K, et al. Toll-like receptor 3 contributes to spinal glial activation and tactile allodynia after nerve injury. J. Neurochem. 2008; 105:2249-2259.
Romero-Sandoval E A, Horvath R J, Deleo J A. Neuroimmune interactions and pain: focus on glial modulating targets. Curr. Opin. Investig. Drugs. 2008; 9:726-734.
Ploesser J, Weinstock L B, Thomas E. Low Dose Naltrexone: Side Effects and Efficacy in Gastrointestinal Disorders.
Tanga F Y, Nutile-McMenemy N, DeLeo J A. The CNS role of Toll-like receptor 4 in innate neuroimmunity and painful neuropathy. Proc. Natl. Acad. Sci. USA. 2005; 102:5856-5861.
   Turk D C, Dworkin R H. What should be the core outcomes in chronic pain clinical trials?. Arthritis Res. Ther. 2004; 6(4):151-4. doi:10.1186/ar1196. PMID 15225358.
Watkins L R, Hutchinson M R, Rice K C, Maier S F (November 2009). “The “Toll” of Opioid-Induced Glial Activation: Improving the Clinical Efficacy of Opioids by Targeting Glia”. Trends in Pharmacological Sciences 30 (11): 581-91. doi:10.1016/j.tips.2009.08.002. PMC 2783351.PMID 19762094.
Webster L R (August 2007). “Oxytrex: an oxycodone and ultra-low-dose naltrexone formulation”. Expert Opin Investig Drugs 16 (8): 1277-83.doi:10.1517/13543784.16.8.1277. PMID 17685875.

1. A method for the treatment of pain in a mammal comprising administration to said mammal a therapeutically effective amount of a composition comprising an opioid/TLR4 antagonist, or a dextro enantiomer of the opioid/TLR4 antagonist or a racemic mixture thereof, or a pharmaceutically acceptable salt or solvate thereof.

2. A method of claim 1 for the treatment of pain in a mammal comprising administration to said mammal a therapeutically effective amount of a composition predominantly comprising a dextro enantiomer of an opioid/TLR4 antagonist or a pharmaceutically acceptable salt or solvate thereof.

3. A method of claim 1 for the treatment of pain in a mammal comprising administration of a composition comprising a therapeutically effective amount of an opioid/TLR4 antagonist. The opioid/TLR4 antagonist is selected from a group consisting of naltrexone, norbinaltorphimine, nalmefene, naloxone, nalorphine, methylnaltrexone, samidorphan, cyprodime, naltrindole, amentoflavone, naltriben, norbinaltorphimine, 6-beta-naltrexol and 6-alpha-natrexol, including all enantiomeric and epimeric forms as well as the appropriate mixtures thereof, as well as pro drugs or metabolites thereof or pharmaceutically acceptable salts or solvates of any thereof.

4. A method of claim 1 for treatment of pain in a mammal comprising administration to said mammal a pharmaceutical composition containing a therapeutically effective amount of naltrexone, naloxone or nalmefene, or a predominantly dextro enantiomeric mixture of naltrexone, naloxone or nalmefene, or a pharmaceutically acceptable salt or solvate of any thereof.

5. A method of claim 1 for treatment of pain in a mammal comprising administration to said mammal a pharmaceutical composition containing a therapeutically effective amount of naltrexone or a pharmaceutically acceptable salt or solvate thereof.

6. A method of claim 1 for treatment of pain in a mammal comprising administration to said mammal a pharmaceutical composition containing a therapeutically effective amount of naloxone or a pharmaceutically acceptable salt or solvate thereof.

7. A method of claim 1 for treatment of pain in a mammal comprising administration to said mammal a pharmaceutical composition containing a therapeutically effective amount of nalmefene or a pharmaceutically acceptable salt or solvate thereof.

8. A method of claim 2 for treatment of pain in a mammal comprising administration to said mammal a pharmaceutical composition containing a therapeutically effective amount of predominantly dextro naltrexone mixture or a pharmaceutically acceptable salt or solvate thereof.

9. A method of claim 2 for treatment of pain in a mammal comprising administration to said mammal a pharmaceutical composition containing a therapeutically effective amount of predominantly dextro naloxone mixture or a pharmaceutically acceptable salt or solvate thereof.

10. A method of claim 2 for treatment of pain in a mammal comprising administration to said mammal a pharmaceutical composition containing a therapeutically effective amount of predominantly dextro nalmefene mixture or a pharmaceutically acceptable salt or solvate thereof.

11. A method of claim 1 for treatment of pain wherein the amount of the opioid/TLR4 antagonist varies from about 0.004 mg/kg to about 4.3 mg/kg, preferably from about 0.004 mg/kg to about 0.71 mg/kg, and most preferably from about 0.004 mg/kg to about 0.21 mg/kg.

12. A method of claim 1 for treatment of pain wherein the amount of naltrexone varies from about 0.004 mg/kg to about 4.3 mg/kg, preferably from about 0.004 mg/kg to about 0.71 mg/kg, and most preferably from about 0.004 mg/kg to about 0.21 mg/kg.

13. A method of claim 2 for treatment of pain wherein the amount of dextro naltrexone varies from about 0.004 mg/kg to about 4.3 mg/kg, preferably from about 0.004 mg/kg to about 0.71 mg/kg, and most preferably from about 0.004 mg/kg to about 0.21 mg/kg.

14. A method of claim 1 for treatment of pain wherein the human dose range of naltrexone, or a pharmaceutically acceptable salt or solvate thereof, varies from about 0.25 mg-50 mg per day, preferably from about 0.25 mg-25 mg per day, most preferably from about 0.25 mg-15 mg per day wherein said dose is formulated into a single dosage form.

15. The method of claim 2 wherein the composition comprises greater than 50% to 60% dextro enantiomer.

16. The method of claim 2 wherein the composition comprises greater than 60% dextro enantiomer.

17. The method of claim 2 wherein the composition comprises greater than 70% dextro enantiomer.

18. The method of claim 2 wherein the composition comprises greater than 80% dextro enantiomer.

19. The method of claim 2 wherein the composition comprises greater than 90% dextro enantiomer.

20. A method of claim 1 for treatment of pain wherein the single fixed dosage form is administered once, twice, three or four times through the day.

21. A method of claim 1 for treatment of pain wherein a therapeutically effective dose is administered systemically, via routes of mucosal, nasal, oral, parenteral, gastrointestinal, topical or sublingual.

22. A method of claim 1 for treatment of pain wherein said composition is in a single dosage form, and said single dosage form is in the form of tablets, lozenges, troches, hard candies, liquid, powders, sprays, creams, salves and suppositories.

23. A method of claim 1 for treatment of pain wherein the pharmaceutical composition is used for the treatment, prevention and reversal of neuropathic pain, back pain, chronic pain, diabetic neuropathic pain, trigeminal neuralgia pain, phantom limb pain, complex regional pain syndrome pain, post herpetic pain, causalgia pain, idiopathic pain, inflammatory pain, cancer pain, postoperative pain, fibromyalgia pain, headache pain, migraine pain, allodynia pain, vulvodynia pain, interstitial cystitis pain, irritable bowel syndrome (IBS), arthritic joint pain and tendinitis.

24. A method of claim 1 wherein the pharmaceutical composition is used for the treatment, prevention and reversal of nociceptive pain with an allodynic component.
